(19) United States
(12) Patent Application Publication
(10) Pub. No.: US 2009/0042778 A1
(43) Pub. Date: Feb. 12, 2009

(54) Methods for Suppressing Neovascularization Using Ephrinb2

(75) Inventors: Kimihiko Fujisawa, Fukuoka (JP); Tatsuro Ishibashi, Fukuoka (JP); Yasuaki Hata, Fukuoka (JP); Tadahisa Kagimoto, Fukuoka (JP)

Correspondence Address: OSHA LIANG L.L.P., TWO HOUSTON CENTER, 909 FANNIN, SUITE 3500, HOUSTON, TX, 77010, US

(73) Assignee: Aqumen Biopharmaceuticals K.K., Fukuoka (JP)

(21) Appl. No.: 11/547,466

(22) PCT Filed: Apr. 5, 2005
(86) PCT No.: PCT/IB2005/002634

§ 371 Date: Oct. 4, 2006

(30) Foreign Application Priority Data
Apr. 5, 2004 (US) 60558999

(51) Int. Cl. A61K 38/17; C12N 5/02; A61P 5/32; A61P 19/02; A61P 27/00; A61P 35/00

(52) U.S. Cl. 514/12; 435/375

(57) ABSTRACT

Methods for inhibiting DNA synthesis, MAP kinase activation, and tube formation of endothelial cells are provided. Also provided are methods for inhibiting angiogenesis and neovascularization, as well as compositions useful for the methods described herein.

### TECHNICAL FIELD

This invention relates to angiogenesis and neovascularization, and more particularly to the use of ephrinB2 therein.

### REFERENCES

Adams, R. H., et al. (2001). The cytoplasmic domain of the ligand ephrinB2 is required for vascular morphogenesis but not cranial neural crest migration. Cell 104(1):57-69.

Adams, R. H., et al. (1999): Roles of ephrinB ligands and EphB receptors in cardiovascular development: demarcation of arterial/venous domains, vascular morphogenesis, and sprouting angiogenesis. Genes Dev 13(3):295-306.

Gale, N. W., et al. (2001). EphrinB2 selectively marks arterial vessels and neovascularization sites in the adult, with expression in both endothelial and smooth-muscle cells. Dev Biol 230(2):151-160.

Gerety, S. S., et al. (1999). Symmetrical mutant phenotypes of the receptor EphB4 and its specific transmembrane ligand ephrinB2 in cardiovascular development. Mol Cell 4(3):403-414.

Kenyon, B. M., et al. (1996). A model of angiogenesis in the mouse cornea. Invest Ophthalmol Vis Sci. 37(8):1625-1632.

Shin, D., et al. (2001). Expression of ephrinB2 identifies a stable genetic difference between arterial and venous vascular smooth muscle as well as endothelial cells, and marks subsets of microvessels at sites of adult neovascularization. Dev Biol 230(2):139-150.

Wang, H. U., et al. (1998). Molecular distinction and angiogenic interaction between embryonic arteries and veins revealed by ephrinB2 and its receptor Eph-B4. Cell 93(5):741-753.

All of the publications, patents and patent applications cited above or elsewhere in this application are herein incorporated by reference in their entirety to the same extent as if the disclosure of each individual publication, patent application or patent was specifically and individually indicated to be incorporated by reference in its entirety.

### BACKGROUND

Angiogenesis is a hallmark of diverse ocular pathological conditions such as age related macular degeneration, diabetic retinopathy and retinopathy of premature. Angiogenic cascade is triggered by a number of mediators and chemokines. For example, endothelial cell receptor tyrosin kinases (RTK), which are associated with the multi-step angiogenesis processes, have been recognized as critical mediators of angiogenesis. The first generation of angiogenic cytokines, including the vascular endothelial cell growth factors (VEGFs), fit well into the concept of sprouting capillaries. More recently, the angiopoietins/Tie2 system has been identified as a vessel assembly and maturation-mediating ligand-receptor system. VEGF/VEGF receptors and angiopoietins/Tie2 receptor families also belong to RTKs.

Eph receptors, the receptors for ephrins, comprise the largest family of tyrosine kinase receptors, consisting of eight EphA and six EphB receptors. Although the Eph receptor tyrosin kinase family represents a new class of RTKs, its role in angiogenesis remains unclear. Originally identified as neuronal pathfinding molecules, knock-out mice and adult ephrinB2-lacZ transgenic mice experiments have identified EphB receptors and ephrinB ligands as crucial regulators of vascular assembly, orchestrating arteriovenous differentiation and boundary formation (Adams et al., 1999; Gale et al., 2001; Shin et al., 2001). Gene-targeting experiments have revealed that ephrinB2 is an early marker of arterial endothelial cells, and its receptor EphB4 reciprocally marks venous endothelial cells in the vertebrate embryo (Wang et al., 1998; Adams et al., 1999; Gerety et al., 1999; Adams et al., 2001). Moreover, endothelial cells in adults maintain their asyrmmetric arteriovenous expression pattern, suggesting that the ephrinB/EphB system plays a role in controlling vascular homeostasis and possesses the possibility to control pathological angiogenesis in adults (Gale et al., 2001; Shin et al., 2001). Thus, it is desirable to determine the mode of action of the ephrins, as well as how these molecules can be used to manipulate the vascular system, particularly in pathological conditions.

### SUMMARY

We investigated the mechanism by which ephrinB2 suppresses angiogenesis. We found that ephrinB2 suppressed endothelial cell (EC) DNA synthesis and both VEGF- and bFGF-induced p44/p42 MAP Kinase activation. In particular, these effects were exerted on both venous and arterial ECs, even though arterial ECs are not known to possess the receptors for ephrinB2. EphrinB2 also inhibited EC tube formation and suppressed bFGF-induced corneal angiogenesis. Our results indicate that targeting ephrinB2/EphB4 and its anti-angiogenic signaling pathway may be beneficial in the treatment of angiogenesis-dependent diseases.

Accordingly, one embodiment of the present invention provides a method for inhibiting DNA synthesis in endothelial cells, comprising contacting the arterial endothelial cells with an effective amount of an ephrinB2. The DNA synthesis in the endothelial cells is preferably induced by VEGF, bFGF or PDGF, and the endothelial cells are preferably arterial endothelial cells.

Also provided is a method for inhibiting p44/p42 MAP kinase activation in endothelial cells, comprising contacting the arterial endothelial cells with an effective amount of an ephrinB2. The p44/p42 MAP kinase activation in the endothelial cells is preferably induced by VEGF, bFGF or PDGF, and the endothelial cells are preferably arterial endothelial cells.

Another embodiment provides a method for inhibiting tube formation from endothelial cell, comprising contacting the arterial endothelial cells with an effective amount of an ephrinB2. The tube formation of the endothelial cells is preferably induced by VEGF, bFGF or PDGF, and the endothelial cells are preferably arterial endothelial cells.

All of these embodiments can be performed in vitro or in vivo. When practiced in vivo, the ephrinB2 can be administered to a mammal comprising the endothelial cells by any method known in the art.

Further provided is a method for inhibiting angiogenesis in a mammal, comprising administering an effective amount of an ephrinB2 to the mammal. The ephrinB2 is preferably a full-length ephrinB2.

Yet another embodiment provides a method for suppressing neovascularization in a mammal, comprising administering an effective amount of an ephrinB2 to the mammal. The ephrinB2 is preferably a full-length ephrinB2.

Still another embodiment provides a method for treating a disease or disorder associated with abnormal neovascularization in a mammal, comprising administering an effective amount of an ephrinB2 to the mammal. The disease or disorder is preferably selected from the group consisting of age-related macular degeneration, ischemic retinopathy, intraocular neovascularization, corneal neovascularization, retinal neovascularization, choroidal neovascularization, diabetic macular edema, diabetic retina ischemia, diabetic retinal edema, diabetic retinopathy, cancers, rheumatoid arthritis and endometriosis.

### BRIEF DESCRIPTION OF THE DRAWINGS

FIG. 1. EphrinB2 inhibits VEGF, bFGF, and PDGF-BB-induced EC proliferation and tube formation.

FIGS. 1A and 1B show the effects of ephrinB2 on VEGF, bFGF, or PDGF-BB-induced DNA synthesis in HAoECs (1A) and HUVECs (1B), respectively. HAoECs or HUVECs were cultured in basal media comprising 10% DMEM at low confluency, and were treated with 200 μg/mL of ephrinB2 or EphB4 either in the presence or absence of VEGF, bFGF, or PDGF-BB (10 ng/mL each) for 24 hours. These experiments were repeated 3 times, and the data shown (mean±SD) are from representative experiments. C: control (vehicle only). B2: ephrinB2. B4: EphB4.

FIG. 1C shows the results of tube formation of epithelial cells in response to VEGF from control, ephrinB2 and EphB4-treated groups at 7 days.

FIG. 2. The effects of ephrinB2 on ERK phosphorylation and receptor autophosphorylation in ECs.

FIG. 2A shows the inhibitory effects of ephrinB2 on ERK phosphorylation in VEGF and bFGF-stimulated HAoECs. p44/p42: total ERK. pp44/pp42: phosphorylated ERK.

FIG. 2B shows that ephrinB2 had no significant effects on autophosphorylation of VEGF-receptor 2 in VEGF-stimulated HAoECs. The receptor (KDR) was imnmunoprecipitated (IP) from cell lysates and blotted with an anti-phosphotyrosine antibody (PY20).

FIG. 3. Administration of ephrin-B2 markedly blocked the neovascularization induced by bFGF. The area with neovascularization in the presence of bFGF was set as 100%.

### DETAILED DESCRIPTION

EphrinB2/EphB4 system plays an important role in vasculogenesis and angiogenesis. We investigated the mechanism by which ephrinB2 suppresses angiogenesis. We found that ephrinB2 suppressed EC DNA synthesis and both VEGF- and FGF2-induced p44/p42 MAP Kinase activation. EphrinB2 also inhibited EC tube formation. We also performed the corneal micropocket assay in mice and quantification of cornea neovascularization. EphrinB2 suppressed bFGF-induced corneal angiogenesis. Our results suggest that targeting ephrinB2/EphB4 and its anti-angiogenic signaling pathway may be beneficial in the treatment of angiogenesis-dependent diseases.

Prior to describing the invention in further detail, the terms used in this application are defined as follows unless otherwise indicated.

### Definitions

The term “ephrinB2”, unless otherwise specified, refers to a polypeptide that (1) shares substantial sequence similarity with a native ephrinB2 or extracellular domain thereof, preferably the native human ephrinB2; and (2) possesses a biological activity of the native ephrinB2 or extracellular domain.

A polypeptide that shares “substantial sequence similarity” with a native molecule is at least about 30% identical with the native molecule at the amino acid level. The polypeptide is preferably at least about 40%, 50%, 60%, 70%, 80%, 85%, 90%, 95%, and most preferably at least about 98% identical with the native molecule at the amino acid level.

The term “percent identity” or “% identity” of an analog or variant with a native molecule refers to the percentage of amino acid sequence in the native molecule which are also found in the analog or variant when the two sequences are aligned. Percent identity can be determined by any methods or algorithms established in the art, such as LALIGN, ClustalW or BLAST.

A polypeptide possesses a “biological activity” of a native ephrinB2 if it is capable of binding to the receptor for the native ephrinB2 or inhibiting EC DNA synthesis, ERK phosphorylation in EC, EC tube formation, angiogenesis or neovascularization. The activity to inhibit EC DNA synthesis, ERK phosphorylation in EC, EC tube formation, angiogenesis or neovascularization can be determined by any methods known in the art, particularly as described in the present application.

A “native” molecule, such as a native ephrinB2, is a molecule that exists without human intervention. However, a native ephrinB2 may also be the extracellular domain of an ephrinB2 that exists without human intervention.

A “full-length” ephrinB2 is an ephrinB2 that contains both an extracellular and an intracellular domain. The full-length ephrinB2 may be native, or it may be an analog or variant of a native ephrinB2.

An “effective amount” is an amount of a substance sufficient to achieve the intended purpose. For example, an effective amount of an ephrinB2 to inhibit DNA synthesis is an amount sufficient, in vivo or in vitro, as the case may be, to result in a reduction in the amount of DNA synthesis. An effective amount of a ephrinB2 to treat a disease or disorder is an amount of the ephrinB2 sufficient to reduce or remove the symptoms of the disease or disorder. The effective amount of a given substance will vary with factors such as the nature of the substance, the route of administration, the size and species of the animal to receive the substance, and the purpose of giving the substance. The effective amount in each individual case may be determined empirically by a skilled artisan according to established methods in the art.

The term “treating” a disease or disorder refers to the reduction or complete removal of the symptoms of a disease or disorder.

### The Effects of EphrinB2 on Epithelial Cells

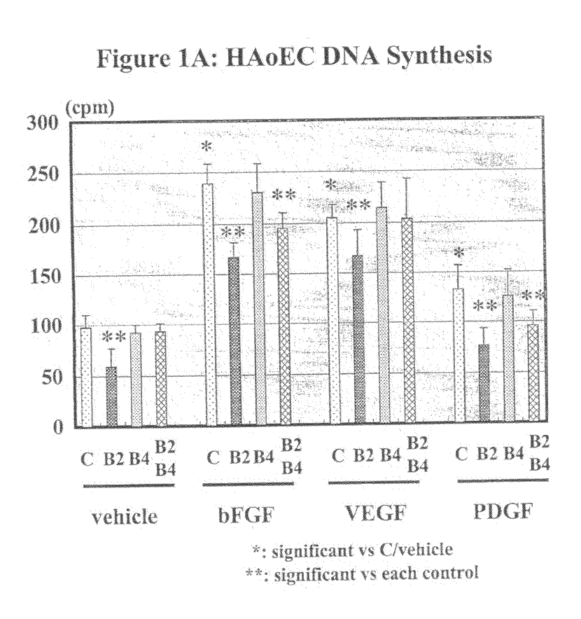

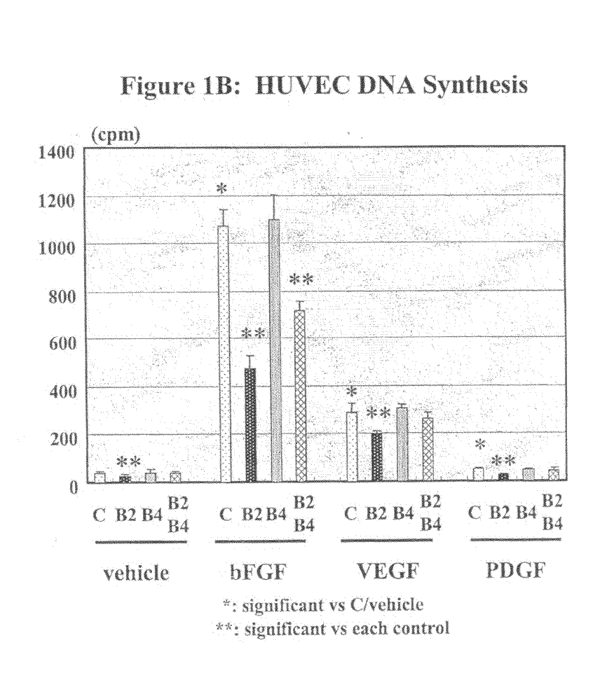

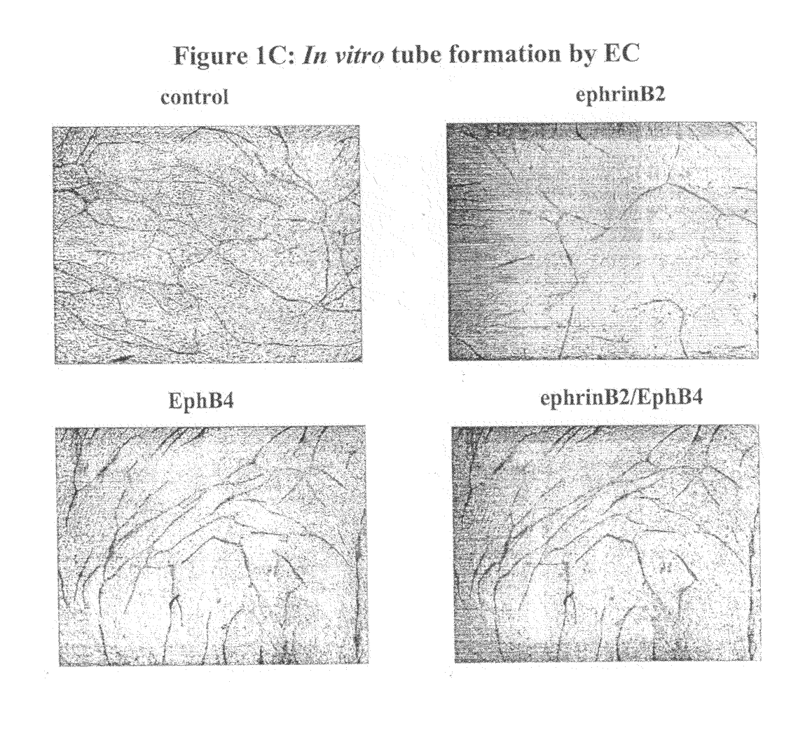

It is known that endothelial cells can be induced to proliferate in response to growth factors, such as VEGF, bFGF, or PDGF-BB. To determine the effect of ephrinB2 on this phenomenon, ephrinB2 was added to arterial or venous epithelial cells in conjunction with VEGF, bFGF, or PDGF-BB (Example 1). The results show that ephrinB2 inhibited DNA synthesis induced by all these stimulants, whereas neither EphB4 nor the combination of ephrinB2 and EphB4 did. As shown in FIG. 1A, the DNA synthesis increase induced by 10 ng/mL of VEGF, bFGF, or PDGF-BB was inhibited by 200 μg/mL of ephrinB2, by 40%, 30%, and 90%, respectively. Virtually identical results were obtained using human umbilical vein endothelial cells (HUVECs) (FIG. 1B). This inhibitory effect of ephrinB2 on DNA synthesis was not caused by apoptosis, as no significant apoptosis was observed.

Accordingly, ephrinB2 is capable of inhibiting EC DNA synthesis that is induced by various stimuli, including VEGF, bFGF and PDGF. It is surprising that DNA synthesis was inhibited in both venous epithelial cells and arterial endothelial cells, because the receptor for ephrinB2 (EphB4) is a marker for venous epithelial cells, while arterial endothelial cells are not known to possess this receptor.

Furthermore, VEGF-induced tube formation was reduced in the ephrinB2 treated group compared with controls at 7 days (FIG. 1C). In contrast, VEGF-induced tube formation was not affected by EphB4 treatment.

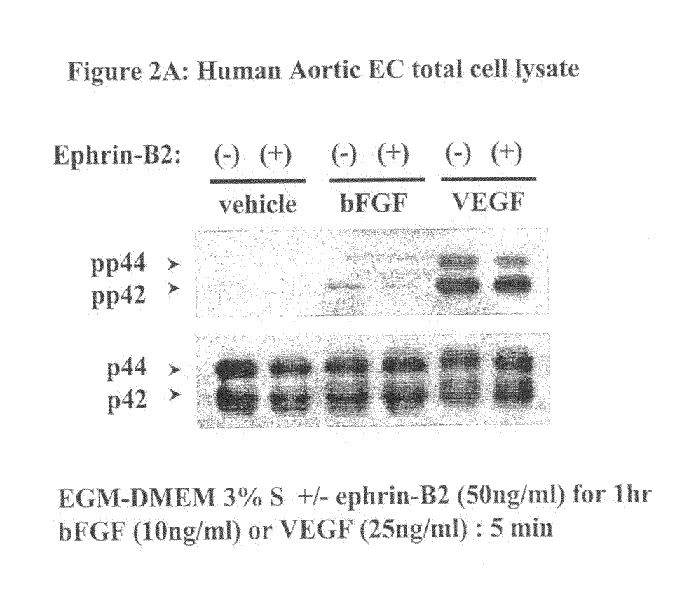

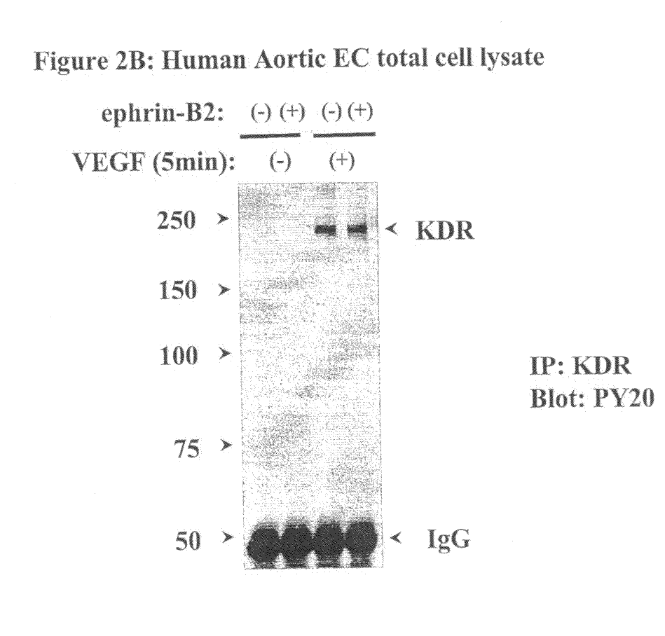

To investigate the mechanism by which ephrinB2 inhibits the growth factor-induced mitogenic response, we examined the effect of ephrinB2 on VEGF or bFGF-stimulated ERK (p42/44) phosphorylation (Example 2). The results indicate that ephrinB2 suppressed both VEGF and bFGF-induced ERK phosphorylation in both arterial and venous endothelial cells. However, ephrinB2 did not inhibit VEGF-receptor 2 autophosphorylation (FIG. 2B), indicating that ephrinB2 does not interfere with signal transduction between VEGF and VEGF-receptor 2 as a mechanism to inhibit VEGF functions. Thus, ephrinB2 can be used to inhibit VEGF or bFGF-induced ERK phosphorylation in either arterial or venous epithelial cells. bFGF is known to be a potent angiogenic factor. Since ephrinB2 is capable of inhibiting EC cell proliferation induced by bFGF, we examined if ephrinB2 can suppress angiogenesis as well (Example 3). Indeed, administration of ephrinB2 markedly blocked the angiogenesis induced by bFGF, but administration of EphB4 showed no effect.

Therefore, ephrinB2 can be used to inhibit angiogenesis and neovascularization. In particular, these results indicate that ephrinB2 is useful in the treatment of diseases or disorders that are associated with angiogenesis or neovascularization, such as age-related macular degeneration, ischemic retinopathy, intraocular neovascularization, corneal neovascularization, retinal neovascularization, choroidal neovascularization, diabetic macular edema, diabetic retina ischemia, diabetic retinal edema, diabetic retinopathy, cancers, rheumatoid arthritis and endometriosis.

The ephrinB2 that is useful in the present invention may be any ephrinB2, including analogs and variants, that possesses the required activity. The ephrinB2 may be full-length, or it may contain the extracellular domain but not the intracellular domain.

### Administration of EphrinB2

Methods for preparing pharmaceutical composition comprising ephrinB2 is well known in the art.

EphrinB2 can be administered systemically, e.g., orally or by IM or IV injection, in admixture with a pharmaceutically acceptable carrier adapted for the route of administration. A variety of physiologically acceptable carriers can be used to administer ephrinB2 and their formulations are known to those skilled in the art and are described, for example, in Remington's Pharmaceutical Sciences (18th edition), ed. A. Gennaro, 1990, Mack Publishing Company, Easton, Pa., and Pollock et al.

EphrinB2 is preferably administered parenterally (e.g., by intramuscular, intraperitoneal, intravenous, intraocular, intravitreal, or subcutaneous injection or implant). Formulations for parenteral administration include sterile aqueous or non-aqueous solutions, suspensions, or emulsions. A variety of aqueous carriers can be used, e.g., water, buffered water, saline, and the like. Examples of other suitable vehicles include polypropylene glycol, polyethylene glycol, vegetable oils, gelatin, hydrogenated naphalenes, and injectable organic esters, such as ethyl oleate. Such formulations may also contain auxiliary substances, such as preserving, wetting, buffering, emulsifying, and/or dispersing agents. Biocompatible, biodegradable lactide polymer, lactide/glycolide copolymer, or polyoxyethylene-polyoxypropylene copolymers may be used to control the release of the active ingredients.

Alternatively, ephrinB2 can be administered by oral ingestion. Compositions intended for oral use can be prepared in solid or liquid forms, according to any method known to the art for the manufacture of pharmaceutical compositions. The compositions may optionally contain sweetening, flavoring, coloring, perfuming, and preserving agents in order to provide a more palatable preparation.

Solid dosage forms for oral administration include capsules, tablets, pills, powders, and granules. Generally, these pharmaceutical preparations contain active ingredient admixed with non-toxic pharmaceutically acceptable excipients.

These may include, for example, inert diluents, such as calcium carbonate, sodium carbonate, lactose, sucrose, glucose, mannitol, cellulose, starch, calcium phosphate, sodium phosphate, kaolin and the like. Binding agents, buffering agents, and/or lubricating agents (e.g., magnesium stearate) may also be used.

Tablets and pills can additionally be prepared with enteric coatings.

Liquid dosage forms for oral administration include pharmaceutically acceptable emulsions, solutions, suspensions, syrups, and soft gelatin capsules.

These forms contain inert diluents commonly used in the art, such as water or an oil medium, and can also include adjuvants, such as wetting agents, emulsifying agents, and suspending agents.

EphrinB2 can also be administered topically, for example, by patch or by direct application to the eye, or by iontophoresis.

EphrinB2 may be provided in sustained release compositions, such as those described in, for example, U.S. Pat. Nos. 5,672,659 and 5,595,760. The use of immediate or sustained release compositions depends on the nature of the disorder being treated. If the disorder consists of an acute or over-acute disorder, treatment with an immediate release form will be preferred over a prolonged release composition. Alternatively, for certain preventative or long-term treatments, a sustained released composition may be appropriate.

EphrinB2 may also be delivered using an implant. Such implants may be biodegradable and/or biocompatible implants, or may be non-biodegradable implants. The implants may be permeable or impermeable to the active agent. An ocular implant may be inserted into a chamber of the eye, such as the anterior or posterior chambers or may be implanted in the schelra, transchoroidal space, or an avascularized region exterior to the vitreous. In a preferred embodiment, the ocular implant may be positioned over an avascular region, such as on the sclera, so as to allow for transcleral diffusion of the drug to the desired site of treatment, e.g., the intraocular space and macula of the eye. Furthermore, the site of transcleral diffusion is preferably in proximity to the macula.

Examples of implants for delivery of ephrinB2 include, but are not limited to, the devices described in U.S. Pat. Nos. 3,416,530; 3,828,777; 4,014,335; 4,300,557; 4,327,725; 4,853,224; 4,946,450; 4,997,652; 5,147,647; 5,164,188; 5,178,635; 5,300,114; 5,322,691; 5,403,901; 5,443,505; 5,466,466; 5,476,511; 5,516,522; 5,632,984; 5,679,666; 5,710,165; 5,725,493; 5,743,274; 5,766,242; 5,766,619; 5,770,592; 5,773,019; 5,824,072; 5,824,073; 5,830,173; 5,836,935; 5,869,079; 5,902,598; 5,904,144; 5,916,584; 6,001,386; 6,074,661; 6,110,485; 6,126,687; 6,146,366; 6,251,090; and 6,299,895, and in WO 01/30323 and WO 01/28474, all of which are incorporated herein by reference.

### Dosage

The amount of active ingredient that is combined with the carrier materials to produce a single dosage will vary depending upon the subject being treated and the particular mode of administration. Generally, ephrinB2 should be administered in an amount sufficient to reduce or eliminate a symptom of a disease.

Dosage levels on the order of about 1 μg/kg to 100 mg/kg of body weight per administration are generally useful in the treatment of neovascular disorders. When administered directly to the eye, the preferred dosage range is about 0.3 mg to about 3 mg per eye. The dosage may be administered as a single dose or divided into multiple doses. In general, the desired dosage should be administered at set intervals for a prolonged period, usually at least over several weeks, although longer periods of administration of several months or more may be needed.

One skilled in the art will appreciate that the exact individual dosages may be adjusted somewhat depending on a variety of factors: the time of administration; the route of administration; the nature of the formulation; the rate of excretion; the particular disorder being treated; the severity of the disorder; and the age, weight, health, and gender of the patient. Wide variations in the needed dosage are to be expected in view of the differing efficiencies of the various routes of administration. For instance, oral administration generally would be expected to require higher dosage levels than administration by intravenous or intravitreal injection. Variations in these dosage levels can be adjusted using standard empirical routines for optimization, which are well known in the art. The precise therapeutically effective dosage levels and patterns are preferably determined by the attending physician in consideration of the above-identified factors.

In addition to treating pre-existing neovascular diseases, ephrinB2 can be administered prophylactically in order to prevent or slow the onset of these disorders. In prophylactic applications, ephrinB2 is administered to a subject susceptible to or otherwise at risk of a particular neovascular disorder. Again, the precise amounts that are administered depend on various factors such as the subject's state of health, weight, etc.

### EXAMPLES

In the examples below, the following abbreviations have the following meanings.

Abbreviations not defined have their generally accepted meanings.

° C.=degree Celsius

hr=hour

min=minute

sec=second

μM=micromolar

mM=millimolar

M=molar

ml=milliliter

μl=microliter

mg=milligram

μg=microgram

DMEM=Dulbecco's modified Eagle's medium

ITS=Insulin-transferrin-selenium

FBS=fetal bovine serum

MEM=modified Eagle's medium

PBS=phosphate buffered saline

VEGF=vascular endothelial cell growth factor

FGF=fibroblast growth factor

PDGF=platelet derived growth factor

SDS=sodium dodecyl sulfate

PAGE=polyacrylamide gel electrophoresis

EC=epithelial cell

HUVEC=Human Umbilical Vein Endothelial Cell

HAoEC=Human Aortic Endothelial Cell

### Materials and Methods

### Reagents

HUVEC were purchased from Clonetics (San Diego, Calif., USA) and maintained in Clonetics EGM medium supplemented with 10% fetal bovine serum (FBS). Endothelial cell growth supplements were also provided by Clonetics. Type I collagen coated dishes were purchased from Iwaki (Japan). [α-³²P] dCTP was purchased from Amersham. Rabbit polyclonal antibody against KDR(sc-504) and Flg were purchased from Santa Cruz Biotechnology (Santa Cruz, Calif., USA).

Cell culture

HUVECs were cultured on type I collagen-coated dishes (Iwaki, Japan) in endothelial growth medium (CLONETICS Corp., San Diego, Calif., USA) at 37° C. in 5% CO₂, 95% air, and the medium was changed every 2-3 days.

### Northern Blot Analysis

Total RNA samples were isolated from cells using acid guanidinium thiocyanate-phenol-chloroform-extraction method and subjected to Northern blot analysis. RNA was fractionated on 1% agarose gel containing 2.2 M formaldehyde, transferred onto a nylon membrane (Nen™ Life Science Products, Inc.), and UV cross-linked at 0.2 J/cm2.

Radioactive KDR or 36B4 cDNA probes were generated using Amersham Multiprime labeling kits and [α-³²P] dCTP. The membrane was hybridized to ³²P-labeled DNA probes in Hybrisol (Amersham, USA) at 42° C. for 16 hours and washed once at room temperature in 2×SSPE (1×SSPE is 0.15 M NaCl plus 0.015M sodium citrate) plus 0.1% sodium dodecyl sulfate (SDS), and twice in 0.5×SSPE plus 0.1% SDS. Messenger RNA levels were quantified by densitometry with Fujix BAS 2500 bioimage analyzer (Fuji Photo Film Co).

### [³H]Thymidine Incorporation—DNA Synthesis of HAoEC and HUBEC

HAoEC and HUBEC were maintained as described. Cells from passages 4 to 5 were used for experiments. ECs were treated for 18 hours in DMEM (Nacalai tesque, Japan) containing 10% FCS with 10 ng/mL of VEGF, bFGF, or PDGF-BB in the presence or absence of the indicated amounts of ephrinB2 and EphB4. The cells were then exposed to [methyl-³H] thymidine (Amersham) at 20 μCi/mL for 6 hours. The cells were trypsinized and retrieved onto glass fiber filters using an automatic cell harvester, and [methyl-³H] thymidine uptake was measured in a direct β counter.

### Endothelial Tube Assay

Collagen gels were formed by mixing together ice-cold gelation solution (10×M199, H₂O, 0.53 M NaHCO₃, 200 mM L-glutamine, type I collagen, 0.1 M NaOH, 100:27.2:50:10:750:62.5 by volume) and cells in 1×basal medium (see below) at a concentration of 3×10⁶ cells/ml at a ratio of 4 volumes gelation solution: 1 volume of cells. After gelation at 37° C. for 30 minutes, the gels were overlaid with 1×basal medium consisting of M199 supplemented with 1% FBS, 1×ITS, 2 mM L-glutamine, 50 μg/ml ascorbic acid, 26.5 mM NaHCO₃, 100 units/ml penicillin, and 100 units/ml streptomycin supplemented with 40 ng/ml bFGF, 40 ng/ml VEGF, and 80 nM PMA. All drugs were added to the 1×basal medium immediately after gelation. To quantitate tube formation, the number of tubes per high power (20×) field was determined 48 hours after addition of the basal medium. A tube was defined as an elongated structure comprised of one or more endothelial cells that exceeded 100 μm in length (long axis). Five independent fields separated by 100 μm optical sections were assessed for each well, and the average number of tubes/20× field determined. Cytoxicity was assessed using a cell proliferation kit II from Boehringer Mannheim.

### Preparation of Protein Samples and Western Blotting

Whole cell lysates, cytosolic or nuclear extracts were isolated from epithelial cells. Western blotting was carried out both with and without immunoprecipitation. Protein samples were separated by sodium dodecyl sulfate-polyacrylamide gel electrophoresis (SDS-PAGE), followed by electrophoretic transfer to nitrocellulose membranes. After blocking with skim milk, the blots were incubated overnight at 4° C. with antibodies against phosphotyrosine or KDR (1:500). After washing, membranes were incubated with horseradish peroxidase-labeled second antibodies (Bio-Rad, Richmond, Calif., USA) (1:3000) for 1 hour at room temperature. Visualization was performed using Amersham enhanced chemiluminescence (ECL) detection system per the manufacturer's instructions.

### Corneal Micropocket Assay in Mice

We essentially performed the corneal micropocket assay in mice and quantification of cornea neovascularization as previously described by Kenyon et al., 1996, with some modifications. Briefly, 0.3 μl of Hydron pellets (IFN Sciences, New Brunswick, N.J., USA) containing 90 ng of human bFGF were prepared and implanted in the corneas of male BALB/c mice. EphrinB2 or EphB4 (100 ng/pellet) was added directly to the bFGF/Hydron solution. The pellet was positioned 1.0 mm from the corneal limbus. After implantation, ofloxacin/eye drops were applied to each eye. After 6 days, the animals were sacrificed and the corneal vessels were photographed. The quantitative analysis of neovascularization in the mouse corneas was performed using the software package NIH Image.

### Statistical Analysis

The experimental data are expressed as means±SD. Statistical significance was assumed when p<0.05 using the Student t-test in normally distributed populations.

### Example 1

### EphrinB2 Inhibits Proliferation and Migration of ECs Stimulated by Growth Factors

Treatment of human aortic epithelial cells (HAoECs) with VEGF, bFGF, or PDGF-BB (10 ng/mL each) increased DNA synthesis by 3-10 fold as compared with the untreated control. EphrinB2 inhibited DNA synthesis stimulated with all these stimulants, whereas neither EphB4 nor ephrinB2+EphB4 did. As shown in FIG. 1A, the DNA synthesis increase induced by 10 ng/mL of VEGF, bFGF, or PDGF-BB was inhibited by 200 μg/mL of ephrinB2, by 40%, 30%, and 90%, respectively. Virtually identical results were obtained using human umbilical vein endothelial cells (HUVECs) (FIG. 1B). No significant apoptotic cells were observed during this incubation period (data not shown). VEGF-induced tube formation was reduced in the ephrinB2 treated group compared with controls at 7 days (FIG. 1C). In contrast, VEGF-induced tube formation was not affected by EphB4 treatment.

Accordingly, ephrinB2 is capable of inhibiting EC DNA synthesis that is induced by various stimuli, including VEGF, bFGF and PDGF. Although the exact mechanism of ephrinB2 is not clear, the decrease in DNA synthesis was not caused by apoptosis, as no significant apoptosis was observed. It is surprising that DNA synthesis was inhibited in both venous epithelial cells and arterial endothelial cells, since the receptor for ephrinB2 (EphB4) is a marker for venous epithelial cells, while arterial endothelial cells are not known to possess this receptor.

Consistent with its effects on DNA synthesis, ephrinB2 is also capable of inhibiting EC tube formation that is induced by VEGF.

### Example 2

### Effects of EphrinB2 on Growth Factor-Induced p42/44 ERK Phosphorylation and Receptor Autophosphorylation

To investigate the mechanism by which ephrinB2 inhibits the VEGF or bFGF-induced mitogenic response on HAoECs, we examined the effect of ephrinB2 on VEGF or bFGF-stimulated ERK (p42/44) phosphorylation by Western analyses. ERK phosphorylation increased by stimulation with 10 ng/mL of VEGF or bFGF. EphrinB2 suppressed both VEGF and bFGF-induced ERK phosphorylation. For example, 200 μg/mL of ephrinB2 inhibited VEGF-induced ERK phosphorylation by 70% (FIG. 2A). Next, we investigated VEGF-receptor 2 (KDR) autophosphorylation in VEGF-stimulated HUVECs. VEGF-receptor 2 autophosphorylation was increased 14-fold by 10 ng/mL of VEGF. EphrinB2 did not inhibit VEGF-receptor 2 autophosphorylation (FIG. 2B). Virtually identical results were obtained using HUVECs (data not shown).

These results thus indicate that ephrinB2 inhibits VEGF or bFGF-induced ERK phosphorylation in either arterial or venous epithelial cells. This effect probably accounts for, at least partially, the activity of ephrinB2 to inhibit VEGF or bFGF-induced proliferation of these cells. However, ephrinB2 does not inhibit autophosphorylation of VEGF-receptor 2. Therefore, ephrinB2 does not interfere with signal transduction between VEGF and VEGF-receptor 2 as a mechanism to inhibit VEGF functions.

### Example 3

### Inhibition of bFGF-induced Angiogenesis by Co-Administration of EphrinB2 in Mouse Corneas

bFGF is known to be a potent angiogenic factor. Since ephrinB2 is capable of inhibiting EC cell proliferation induced by bFGF, we examined if ephrinB2 can suppress angiogenesis as well.

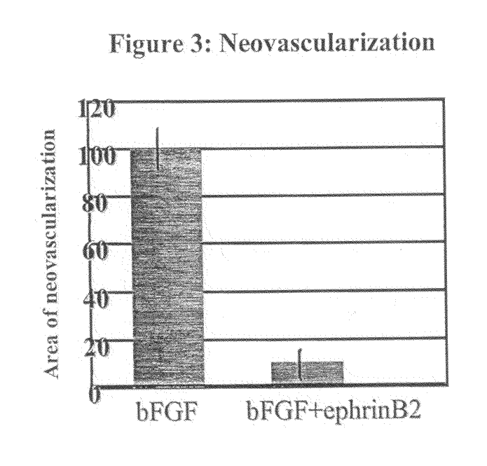

We implanted a pellet of Hydron that had been impregnated with human bFGF into the corneas of mice. Six days after pellet implantation, we examined the outgrowth of new blood vessels. bFGF dramatically induced angiogenesis in mouse corneas. We further examined the effects of ephrinB2 and Eph B4 on bFGF-induced corneal neovascularization. Administration of ephrinB2 markedly blocked the angiogenesis induced by bFGF, however administration of EphB4 showed no effect. Quantitative analysis also demonstrated that bFGF-induced corneal neovascularization was completely inhibited by ephrinB2 (FIG. 3).

Therefore, ephrinB2 can be used to inhibit angiogenesis and neovascularization. In particular, these results indicate that ephrinB2 is useful in the treatment of diseases or disorders that are associated with angiogenesis or neovascularization, such as age-related macular degeneration, ischemic retinopathy, intraocular neovascularization, corneal neovascularization, retinal neovascularization, choroidal neovascularization, diabetic macular edema, diabetic retina ischemia, diabetic retinal edema, diabetic retinopathy, cancers, rheumatoid arthritis and endometriosis.

A number of embodiments of the invention have been described. Nevertheless, it will be understood that various modifications may be made without departing from the spirit and scope of the invention.

1. A method for inhibiting DNA synthesis in arterial endothelial cells, comprising contacting the arterial endothelial cells with an effective amount of an ephrinB2.

2. The method of claim 1 wherein the DNA synthesis is induced by VEGF, bFGF or PDGF.

3. (canceled)

4. (canceled)

5. A method for inhibiting tube formation from arterial endothelial cells, comprising contacting the arterial endothelial cells with an effective amount of an ephrinB2.

6. The method of claim 5 wherein the tube formation is induced by VEGF, bFGF or PDGF.

7. The method of claim 1 wherein the ephrinB2 comprises the extracellular domain but not the cytoplasmic domain of a native ephrinB2.

8. The method of claim 1 wherein the ephrinB2 is administered to a mammal comprising the arterial endothelial cells.

9. The method of claim 7 wherein the ephrinB2 is administered to a mammal comprising the arterial endothelial cells.

10. A method for inhibiting DNA synthesis in venous endothelial cells, comprising contacting the venous endothelial cells with an effective amount of an ephrinB2.

11. The method of claim 10 wherein the DNA synthesis is induced by VEGF, bFGF or PDGF.

12. A method for inhibiting p44/p42 MAP kinase activation in venous endothelial cells, comprising contacting the venous endothelial cells with an effective amount of an ephrinB2.

13. The method of claim 12 wherein the p44/p42 MAP kinase activation is induced by VEGF, bFGF or PDGF.

14. A method for inhibiting tube formation from venous endothelial cells, comprising contacting the venous endothelial cells with an effective amount of an ephrinB2.

15. The method of claim 14 wherein the tube formation is induced by VEGF, bFGF or PDGF.

16. The method of claim 10 wherein the ephrinB2 comprises the extracellular domain but not the cytoplasmic domain of a native ephrinB2.

17. The method of claim 10 wherein the ephrinB2 is administered to a mammal comprising the venous endothelial cells.

18. The method of claim 16 wherein the ephrinB2 is administered to a mammal comprising the venous endothelial cells.

19. A method of suppressing ocular neovascularization in a mammal, comprising administering to the mammal an effective amount of an ephrinB2.

20. A method of suppressing choroidal neovascularization in a mammal, comprising administering to the mammal an effective amount of an ephrinB2.

21. A method for treating a disease or disorder associated with abnormal ocular neovascularization in a mammal, comprising administering to the mammal an effective amount of an ephrinB2.

22. The method of claim 21 wherein the disease or disorder is selected from the group consisting of age-related macular degeneration, ischemic retinopathy, intraocular neovascularization, corneal neovascularization, retinal neovascularization, choroidal neovascularization, diabetic macular edema, diabetic retina ischemia, diabetic retinal edema, and diabetic retinopathy.

23. The method of claim 22 wherein the disease or disorder is age-related macular degeneration.

24. The method of claim 19 wherein the ephrinB2 comprises the extracellular domain but not the cytoplasmic domain of a native ephrinB2.
